(19) United States
(12) Patent Application Publication
(10) Pub. No.: US 2009/0325287 A1
(43) Pub. Date: Dec. 31, 2009

(54) Methods for Obtaining High Viable Cell Density in Mammalian Cell Culture

(76) Inventors: Haimanti Dorai, Radnor, PA (US); Yun Seung Kyung, Radnor, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/483,626

(22) Filed: Jun. 12, 2009

Related U.S. Application Data
(60) Provisional application No. 61/061,233, filed on Jun. 13, 2008.

(51) Int. Cl. C12N 5/06

(52) U.S. Cl. 435/326; 435/325; 435/358

(57) ABSTRACT

Methods for increasing viability in fed batch eukaryotic cell culture are disclosed.

### CROSS-REFERENCE TO RELATED APPLICATIONS

This application claims priority to U.S. Provisional Application Ser. No. 61/061,233, filed 13 Jun. 2008, the entire contents of which is incorporated herein by reference in its entirety.

### FIELD OF THE INVENTION

The present invention relates to methods for achieving high viable cell density and extended culture longevity in fed batch cell culture by using a high glucose feed. The methods are useful for increasing production of secreted proteins of interest.

### BACKGROUND OF THE INVENTION

Mammalian cell culture is the system of choice for many recombinant protein production processes due to its ability to produce proteins with proper post-translational modifications. With increasing manufacturing demand, there is a strong motivation to improve process efficiency by increasing product yield. Attaining grams per liter production levels of biotherapeutics or other proteins in commercial production processes relies upon the optimization of both mammalian cell culture and engineering methods. Inherent in current high density, protein-free mammalian cell cultures is the problem of cell death of which apoptosis can account for up to 80% in a typical fed-batch bioreactor, induced in response to conditions such as nutrient and growth factor deprivation, oxygen depletion, toxin accumulation, and shear stress (Goswami et al., Biotechnol Bioeng 62:632-640 (1999)). Apoptosis limits the maximum viable cell density, accelerates the onset of the death phase and potentially decreases heterologous protein yield (Chiang and Sisk, Biotechnol Bioeng 91:779-792 (2005); Figueroa et al., Biotechnol Bioeng.73:211-222 (2001), Metab Eng 5:230-245 (2003), Biotechnol Bioeng 85:589-600 (2004); Mercille and Massie, Biotechnol Bioeng 44:1140-1154 (1994)).

Apoptosis is a result of a complex network of signaling pathways initiating from both inside and outside the cell, culminating in the activation of cysteine aspartate proteases (caspases) that mediate the final stages of cell death. See FIG. 1. Various methods of apoptosis prevention have been used to maintain cell viability during extended production runs in mammalian cell culture (Arden and Betenbaugh, Trends Biotechnol 22:174-180 (2004); Vives et al., Metab Eng 5:124-132 (2003)). Altering the extracellular environment through media supplementation of growth factors, hydrolysates, and limiting nutrients has led to increased protein production and decreased apoptosis (Burteau et al., In Vitro Cell Dev Biol Anim. 39:291-296 (2003); Zhang and Robinson, Cytotechnology 48: 59-74 (2005)). Other researchers have turned to chemical and genetic strategies to inhibit the apoptotic signaling cascade from within the cell (Sauerwald et al., Biotechnol Bioeng 77:704-716 (2002), Biotechnol Bioeng 81:329-340 (2003]).

Researchers have found that over-expression of genes found upregulated in cancer cells can prolong viability in cells grown in bioreactors by preventing apoptosis upstream of caspase activation (Goswami et al., supra; Mastrangelo et al., Trends Biotechnol 16:88-95 (1998); Meents et al., Biotechnol Bioeng 80:706-716 (2002); Tey et al., J Biotechnol 79:147-159 (2000) and Biotechnol Bioeng 68:31-43 (2000). Upregulation of these proteins in production cell lines effectively suppressed apoptotic signaling within the cell, thereby limiting cell death in order to maintain viability and increase biotherapeutic production in some cases.

Also inherent in high density, protein-free mammalian cell cultures is the problem of waste accumulation and its detrimental effect on cell growth. The two most common cell culture waste products are lactate and ammonia. Numerous strategies have been devised to address the accumulation of excessive lactate build-up including 1) maintaining low medium glucose concentrations (Kurokawa et al., Biotechnol Bioeng 44:95-103 (1994); Xie and Wang, Biotechnol Bioeng 43:1175-1189 (1993); Zhang et al., J Chem Technol Biotechnol 79:171-181 (2004); Zhou et al., Biotechnol Bioeng 46:579-587 (1995)); 2) feeding alternative sugars, including fructose (Martinelle et al., Biotechnol Bioeng 60:508-517 (1998), Altamirano et al., J Biotechnol 110:171-179 (2004) and J Biotechnol 125:547-556 (2006), Walschin and Wu, J Biotechnol 131:168-176 (2007); 3) partially knocking out lactate dehydrogenase (LDH) expression by homologous recombination or siRNA technology; 4) over-expression of pyruvate carboxylase; 5) use of dichloracetate (DCA), a pyruvate dehydrogenase (PDH) activator (via PDH kinase inhibition); 6) oxamic acid, an LDH competitive inhibitor; and 7) removal through perfusion (US Pat. Appl. Publ. No. 2009/0042253 A1).

Originally, the exclusive function of many of the apoptotic pathway proteins was believed to be binding at the mitochondrial membrane and regulating apoptosis through modulation of mitochondria permeability. Recent findings have shown that key proteins involved in apoptotic signaling interact with and have effects on proteins that control metabolism and energy homeostasis in the cell. See Majors et al., Metab Eng 9:317-326 (2007) for a review; and White et al., Nat Cell Biol 7:1021-1028 (2005). In a recent study, microarray analysis of CHO cells over-expressing Bcl-XL show that lactate dehydrogenase, a key enzyme in gluconeogenesis, is up-regulated.

Certain cells and viruses produce anti-apoptotic genes that function in the mitochondrial apoptotic pathway. These can be divided into three groups, namely 1) those that act early in the pathway, e.g., members of the Bcl-2 family of proteins; 2) those that act mid-pathway to disrupt or inhibit the apoptosome complex, e.g., Aven and 3) those that act late in the pathway, e.g., caspase inhibitors such as XIAP. The functionality of the majority of these genes have been studied by over-expressing them in mammalian expression systems, and in some cases the effect of combined over-expression of two or more genes, each derived from a different part of the pathway has been determined. Examples include 1) the additive effect of Bcl-XL and a deletion mutant of XIAP (XIAPΔ) in CHO cells (Figueroa et al., Metab. Eng. 5:230-245 (2003)); 2) E1B-19K and Aven in BHK cells (Nivitchanyong et al., Biotechnol Bioeng 98:825-841 (2007)) and 3) Bcl-XL, Aven and XIAPΔ (Sauerwald et al., supra, (2003); Sauerwald et al, Biotechnol Bioeng 94:362-369 (2006)). However, in these studies, the effect of the anti-apoptotic genes on the cellular metabolic state of the cell was not examined. Accordingly, a need exists in mammalian cell culture systems to optimize nutrient consumption and metabolite accumulation conditions to achieve increased viable cell density, longevity and productivity.

### BRIEF DESCRIPTION OF THE DRAWINGS

FIG. 1 shows mitochondrial apoptosis pathway components.

FIGS. 2A-2C show the characterization of apoptotic^{R} cell lines.

FIG. 3 shows confirmation of apoptotic^{R} in EA167 and EAX197 by analysis by flow cytometry.

FIGS. 4A-4C show growth profiles of apoptotic^{R} cell lines generated from transfection of E1B-19K, E1B-19K+Aven and E1B-19K+Aven+XIAPΔ. Shake flask batch cultures of control (•), E64 (♦), EA167 (▪) and EAX197 (▾) were monitored for viable cell density and viability.

FIGS. 5A-5C show metabolite profiles of apoptotic^{R} cell lines generated from transfection of E1B-19K+AVEN±XIAPΔ. Shake flask batch cultures of control (•), EA167 (▪) and EAX197 (▾) cell lines were monitored for various metabolites.

FIGS. 6A and 6B show daily lactate replenishment of apoptotic^{R} cell lines. A) (•), control and EA167: □, un-fed and ▪, lactate-fed) and B) EAX197 (Δ, un-fed and ▾, lactate-fed).

FIGS. 7A-7G show growth and metabolic profiles of control and apoptotic^{R} cell lines EA167 and EAX197 in the presence or absence of lactate feeding. Control (•), EA167 (□, un-fed and ▪, lactate-fed), EAX197 (▴, un-fed and ▾, lactate-fed).

FIGS. 8A-8G show profiles of amino acids depleted by apoptotic^{R} cell lines upon lactate feeding. Control (•), EA167 (□, un-fed and ▪, lactate-fed), EAX197 (▴, un-fed and ▾, lactate-fed).

FIGS. 9A-9D show growth profiles and lactate accumulation profiles of control and apoptotic^{R} cell lines EAX 197 cultured in high concentrations of glucose in a custom medium formulation. Apoptotic^{R} cell line EAX197 (▪); control cell line (♦).

FIGS. 10A and 10B show antibody titers of cell lines generated from EAX197 and control host cell lines cultured in custom media formulation containing 30 mM or 60 mM glucose. Apoptotic^{R} cell line EAX197 (▪); control cell lines (□).

FIGS. 11A and 11B show VCD and titer of a cell line containing the apoptotic^{R} gene Bcl-2 with high and low cell seeding densities and media containing 30 mM or 60 mM glucose.

### SUMMARY OF THE INVENTION

One aspect of the invention is a method for obtaining high viable cell density in a fed batch eukaryotic cell culture comprising the steps of:

a) culturing a eukaryotic cell line expressing one or more heterologous apoptotic-resistant (apoptotic^{R}) genes and one or more genes of interest; and

b) maintaining a high glucose media feed during the exponential and stationary phases of the cell culture.

Another aspect of the invention is a method of increasing production of secreted proteins in a fed batch eukaryotic cell culture comprising the steps of:

a) culturing a eukaryotic cell line expressing one or more heterologous apoptotic-resistant (apoptotic^{R}) genes and one or more genes of interest; and

b) maintaining a high glucose media feed during the exponential and stationary phases of the cell culture.

### DETAILED DESCRIPTION OF THE INVENTION

All publications, including but not limited to patents and patent applications, cited in this specification are herein incorporated by reference as though fully set forth.

As used herein, the term “fed batch cell culture” means a cell culture process which is based on feeding of a growth limiting nutrient substrate to the culture. The fed-batch strategy is typically used in bio-industrial processes to reach a high cell density in a bioreactor. However, the addition of a nutrient such as glucose results in formation of metabolic waste products such as lactate and ammonia. Concentrations of 18 mM lactate (Kurano et al., 1990) and 8 mM ammonia (Hansen and Emborg, 1994) have been reported as inhibitory for eukaryotic cell growth.

Numerous strategies have been devised to address the accumulation of excessive lactate build-up in batch-fed cell culture and are discussed above. In the present invention, an alternative approach to reducing lactate concentration and increasing viable cell density and viability is presented. The method involves over-expressing one or more anti-apoptotic genes in a host cell line. The resultant apoptotic-resistant cell lines stimulate mitochondrial respiration and accumulate less lactate. Therefore, these cell lines can thrive when fed with high concentrations of glucose. Consequently, cultures of these apoptotic-resistant cell lines can reach high viable cell density. These cell lines also possess extended longevity due to their apoptotic-resistant nature.

The methods of the invention are useful for increasing viable cell density and viability in fed batch cell culture, such as Chinese Hamster Ovary (CHO), myeloma or hybridoma cell cultures. In particular, the methods of the invention are useful for increasing the integrated viable cell count (IVCC) of CHO cell cultures. The method of the invention comprises the steps of culturing a cell line expressing one or more heterologous apoptotic-resistant (apoptotic^{R}) genes and one or more genes of interest; and maintaining a high glucose media feed during the exponential and stationary phases of the cell culture. These lines are superior hosts for the development of production cell lines expressing proteins of interest such as peptides, peptide fusions, growth factors, hormones, antibodies, designed ankyrin repeat proteins (DARPins) and other polypeptides useful for therapeutic, diagnostic or research purposes. CHO cell lines useful in the method of the invention include CHO-K1 (Invitrogen, Carlsbad, Calif.) and CHOK1SV (Lonza Biologics, Slough, UK). Myeloma lines useful in the method of the invention include NS0 and Sp2/0.

The cell lines useful in the method of the invention express one or more heterologous anti-apoptosis genes. In particular, the genes encoding E1B19K (SEQ ID NOs: 1 and 2) and Aven (SEQ ID NOs: 3 and 4) are useful. The gene encoding XIAPΔ (SEQ ID NOs: 5 and 6) can also be used. These anti-apoptotic genes are representatives of the early, mid- and late stages of the apoptotic signaling pathway, respectively. Further, the gene encoding Bcl-2Δ can also be used (SEQ ID NOs: 7 and 8). Expression of the anti-apoptosis genes can be achieved by transfection techniques known to those skilled in the art. It is contemplated that other anti-apoptosis genes from these stages of the apoptotic signaling pathway, such as MDM2 (SEQ ID NOs: 9 and 10) and Bcl-XL (SEQ ID NOs: 11 and 12), would also be useful in the methods of the invention.

In the present invention, the use of cell lines expressing heterologous anti-apoptotic genes allows these cell lines to secrete less lactate or alternatively, to consume accumulated lactate, to reach integrated viable cell count (IVCC) values about two-fold higher than control cell lines while a high glucose feed is maintained in the culture. By increasing the viable cell densities of production cell lines in culture, the yields of products obtained from a bioreactor run are increased. Such enhanced productivities can result in lower production costs for complex biologics and at the same time, generate product of superior quality due to the absence of cell lysis of the non-viable cells, as lysed cells release proteases that degrade product. Accordingly, these lines are superior hosts for the development of production cell lines expressing a protein or proteins of interest.

In the method of the invention, the ability of the apoptotic^{R} cell lines to consume or produce less lactate that would otherwise accumulate as waste provides for culture strategies to allow growth under conditions of high glucose as well as following glucose depletion. In different embodiments, the methods of the invention provide for increased peak viable cell density, increased longevity, increased secreted protein titer, increased integrated viable cell count, reduced cellular calcium flux and increased mitochondrial membrane potential in batch fed cell culture. Furthermore, strategies of fed-batch cultivation that have been used to limit high levels of toxic lactate and ammonia are unlikely to be necessary for high productivity from these cell lines.

Following transfection, the site of chromosomal integration of an anti-apoptotic transgene can dictate the level of its expression. Moreover, when multiple anti-apoptotic genes are transfected, the level of expression of a particular anti-apoptotic transgene can impact the activity of other anti-apoptotic proteins the cell possesses since many of these proteins interact directly or indirectly to bring about physiological changes in the cell. Thus the quantitative contribution of each anti-apoptotic gene towards the overall apoptotic^{R} characteristics of a cell line with multiple genes can be difficult to determine due to synergistic effects of the apoptotic^{R} genes. Additionally, significant clone-to-clone variations exist with respect to apoptotic^{R} characteristics even though all of them have been transfected with an identical set of transgenes. What is clear from the results presented in the Examples below is that each anti-apoptotic gene tested offered an incremental positive value towards improving the apoptosis^{R} characteristics of the host cell line into which it was transfected. Within the above constraints, triple transfectants were generally superior to double transfectants, which in turn were superior to transfectants over-expressing only one anti-apoptotic transgene.

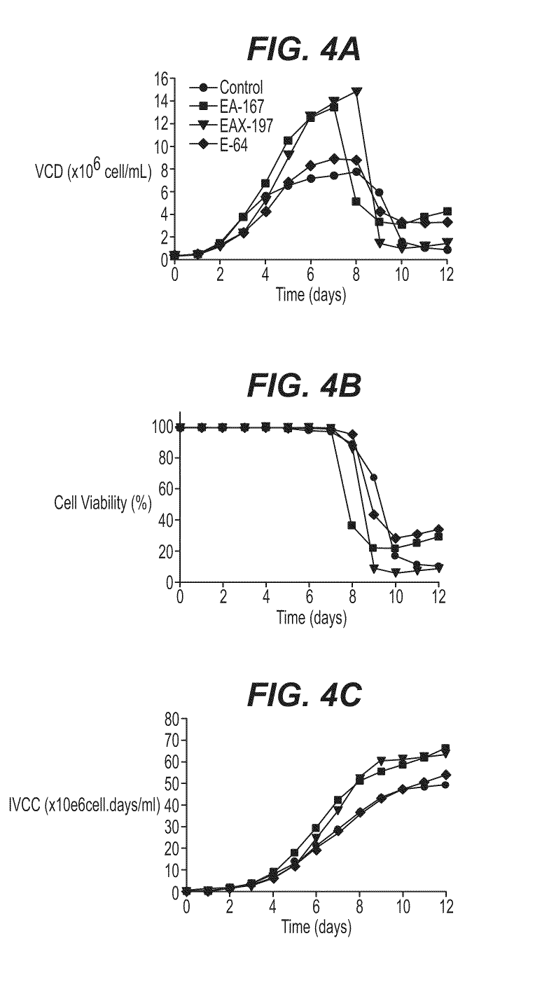

Consequently, following transfection with appropriate expression vectors, clones were obtained with variable levels of expression of E1B-19K, Aven, and XIAPΔ. The expression level of E1B-19K was relatively low and rare clones that had high expression level of E1B-19K were either unstable or had poor growth properties (data not shown). In the Examples below, only limited protection against cell death was observed in CHO cells expressing E1B-19K alone (FIG. 4).

Contrary to results obtained with cell lines expressing E1B-19K alone, data presented in the Examples below demonstrate that CHO K1 cell lines co-expressing Aven (EA167) or Aven and XIAPΔ (EAX197) exhibited improvements in viability, cell density and IVCC. The results presented in the Examples below further demonstrate that the expression of two anti-apoptosis genes in CHO K1 leads to a significant reduction in caspase activity and improved mitochondrial membrane potential in the presence of insults including staurosporine and extended batch cultures.

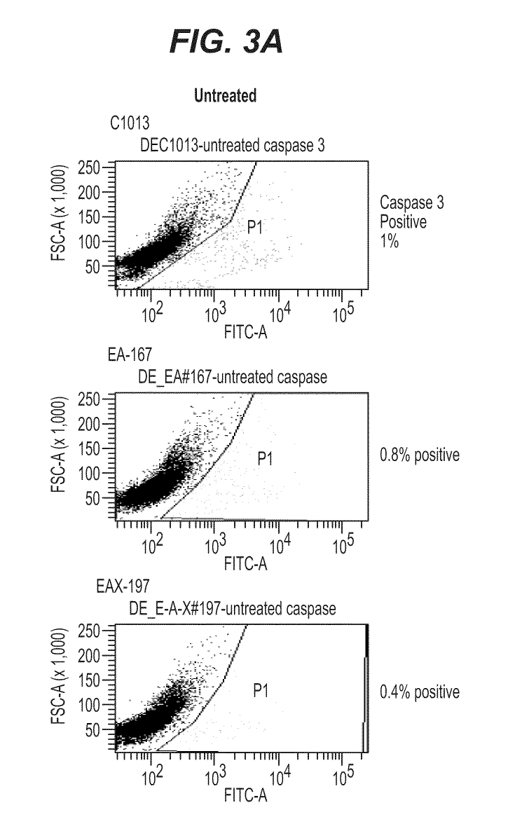

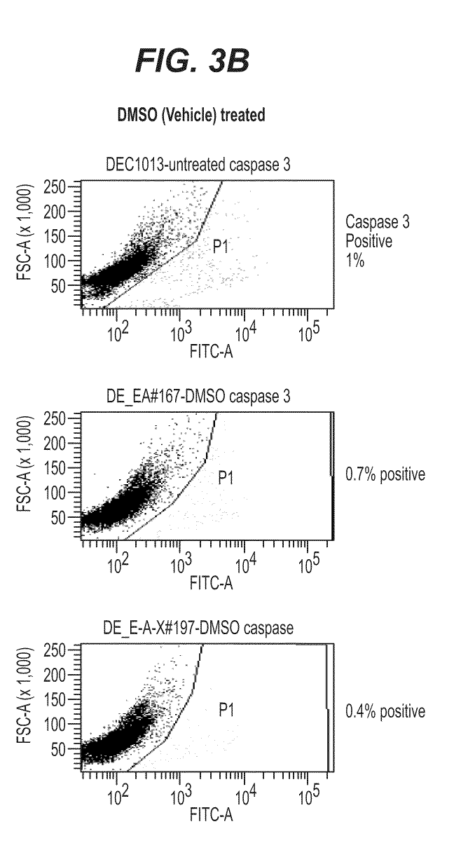

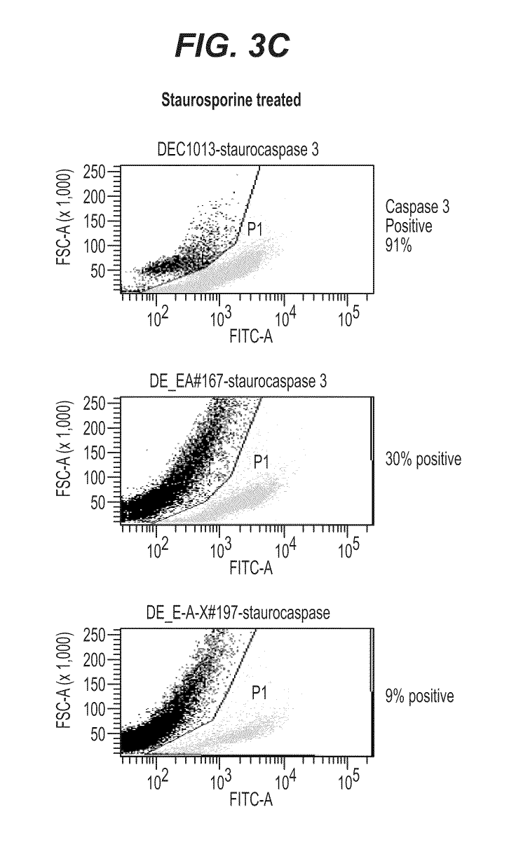

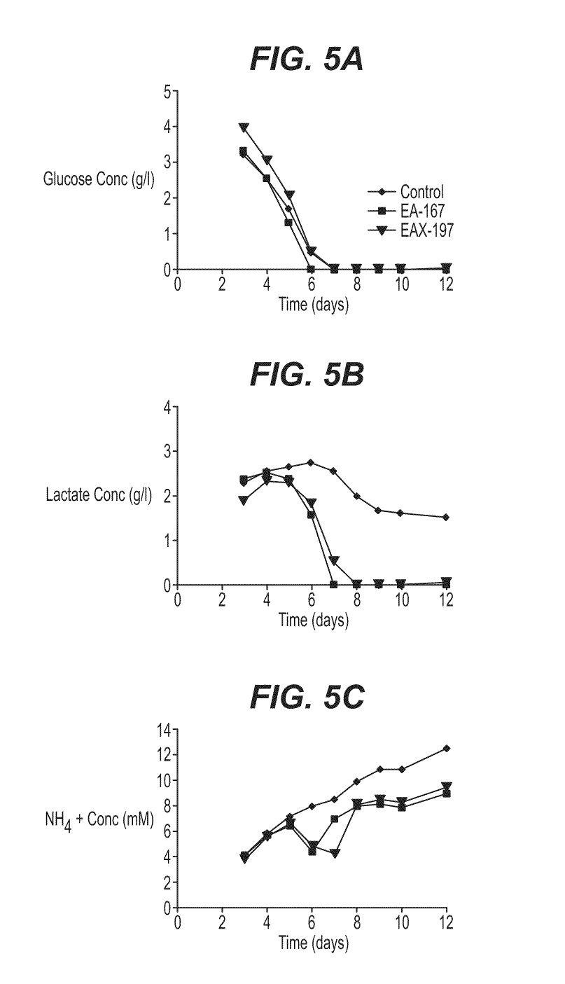

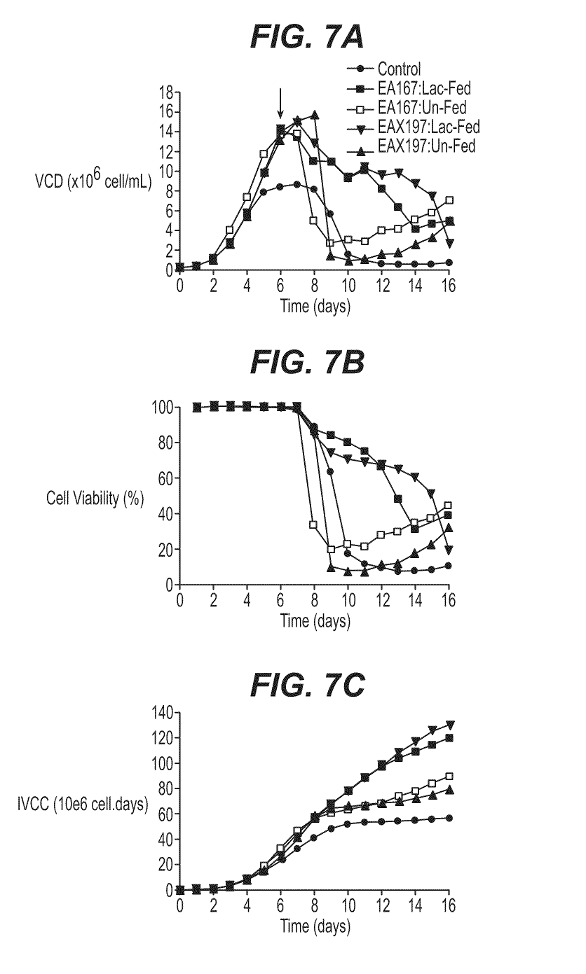

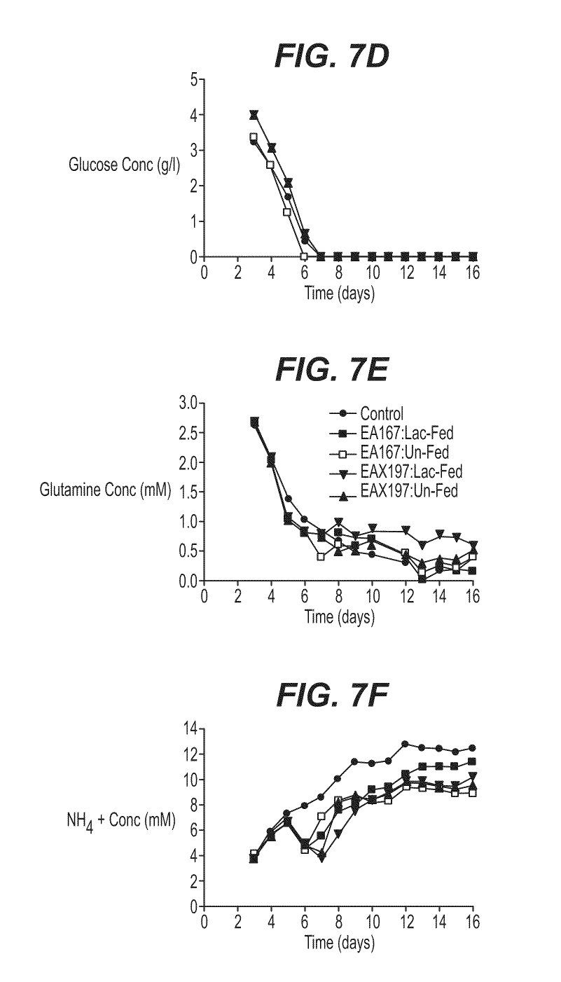

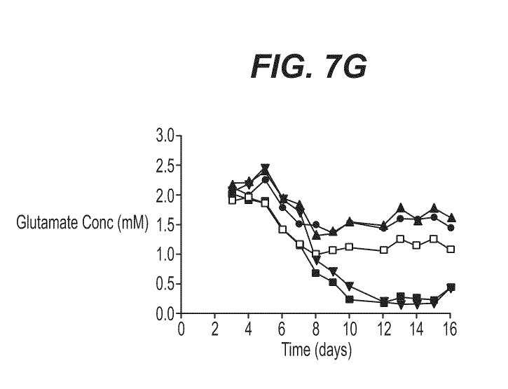

In the Examples below, the expression of a combination of E1B-19K, which is a functional homolog of Bcl-XL, plus Aven, and XIAPΔ was examined for effects on delaying cell death. The level of XIAPΔ expression in these transfected cells was not high relative to the endogenous wild type XIAP protein already present in the cell, perhaps due to the requirement for transfecting two plasmids in this system. Nonetheless, the addition of XIAPΔ in EAX197 (in addition to Aven and E1B-19K) provided a consistent enhancement in the maximum viable cell density in both unsupplemented (FIGS. 5 and 7) and lactate supplemented cultures (FIG. 7). Indeed, the EAX197 cell line fed with lactate was able to sustain high viabilities at or above nearly 60% for 14 days, which was two days longer than the EA167 cells and four days longer than the control culture. The inclusion of XIAPΔ likely increases the cell's resistance to caspase activation and this is verified in FIG. 3 by the very low caspase activity of EAX197 even after treatment with staurosporine. Furthermore, the use of a mutant of XIAP (XIAPΔ) provides enhanced apoptosis resistance relative to the wild type XIAP protein. Thus, the protective capacity of the deletion mutant of XIAP (XIAPΔ) may be significant even if the expression of the protein is not high in these CHO lines.

In the Examples below, the role of one or more anti-apoptotic gene(s) in cell metabolism was investigated. Nutrient consumption and metabolite production were determined in the apoptotic^{R} cell lines expressing E1B-19K in conjunction with Aven and/or XIAPΔ. In particular, the accumulation of the two most common cell culture waste products (i.e., lactate and ammonia) were compared in apoptotic^{R} cell lines to that of the control cell line. In the data presented below, it was found that in shake-flask batch cultures in CD-CHO medium, on day 6-day 7 post-seeding, the control cell line accumulated 2.8 g/L (31 mM) lactate and 12 mM ammonia (FIG. 5), which are well above the concentrations of 18 mM of lactate (Kurano et al., supra) and 8 mM of ammonia (Hansen and Emborg, supra) reported as inhibitory for cell growth. These levels may be part of the reason for loss in viability seen by day-8 post-inoculation in CHO control cell line (FIG. 4).

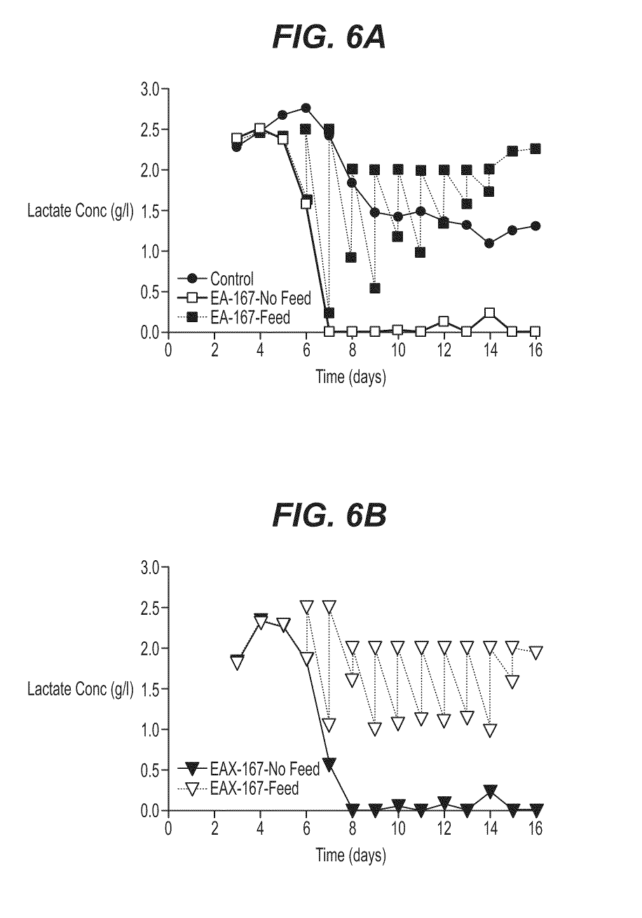

While the data presented below show that apoptotic^{R} cell lines accumulate some lactate early, these cell lines began to consume lactate during the exponential phase and continued to increase in VCD well beyond that of the control cultures. Either the ability to consume lactate immediately following glucose exhaustion or an increase in apoptosis inhibition or both factors contributed to the continued cell growth. Furthermore, the apoptotic^{R} cells only entered the stationary phase when the endogenous lactate was exhausted. In order to determine if the consumption of lactate was a general characteristic of these engineered cell lines, exogenous lactate was added to the apoptosis^{R} cultures to replenish the depleted lactate (FIG. 6). The results presented in the Examples below show that the apoptotic^{R} cultures consumed the added lactate and maintained high VCD and viabilities for four to seven additional days as compared to control cultures (for un-fed and lactate-fed cultures, respectively; FIG. 7A and B). An added process benefit is that lower levels of lactate in cultures of apoptotic^{R} cell lines will result in lower osmolarity and higher pH, both of which are highly desirable for superior product quality.

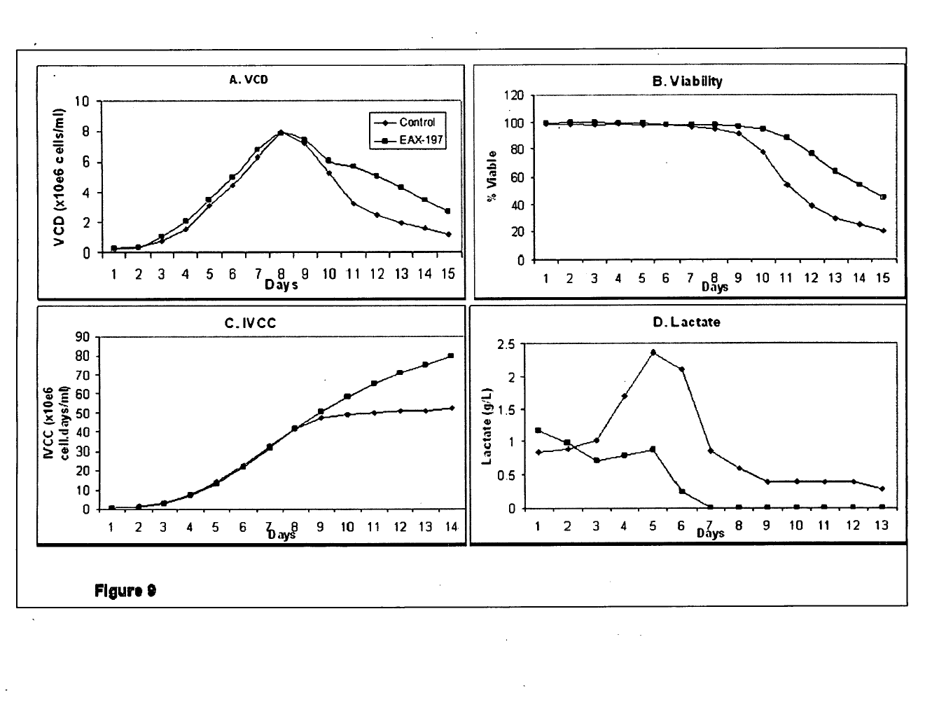

Previous researchers have detected lactate consumption in hybridoma, NS0 myeloma and CHO cultures (deZengotita et al., 2000; Zhou et al., 1995 and 1997; Burky et al., 2007; Pascoe et al., 2007). However, these cells typically exhibit lactate production in the exponential phase and transition to consumption in the stationary phases to suggest that lactate can represent both an intermediate by-product and a carbohydrate fuel source (Burky et al., 2007; Brooks et al., 1985). In the results presented below, the control CHO cultures appeared to consume some lactate in the stationary phase, particularly in the ‘high’ glucose medium (FIG. 9), as seen in the previous studies. In contrast, the apoptotic^{R} cells exhibited a dramatically different profile in which lactate was consumed during the exponential phase and the cells only entered the stationary phase when the exogenous lactate was depleted.

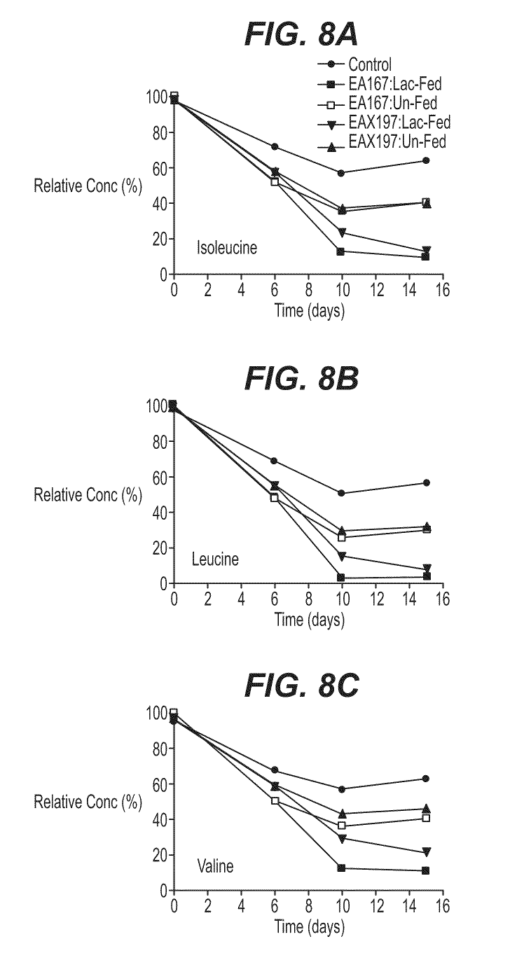

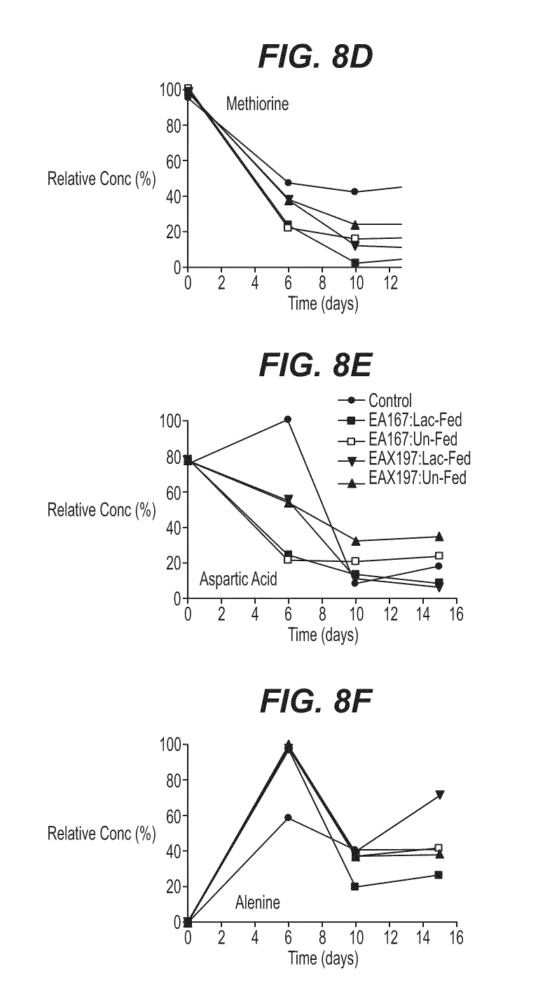

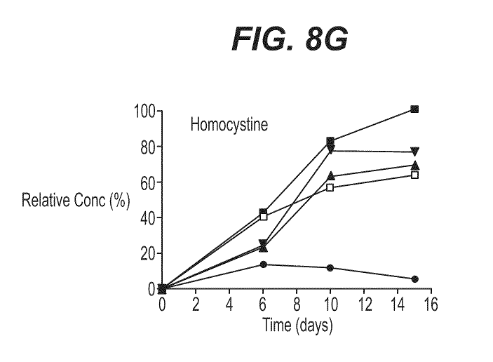

The conversion of pyruvate to lactate, catalyzed by lactate dehydrogenase (LDH), is reversible but strongly favors lactate formation with an equilibrium constant of 3.6×10⁴/M. However, when glycolysis is unable to keep up with tricarboxylic acid (TCA) demand, lactate can be converted back to pyruvate by one of the several isozymes of LDH favoring that reaction. The lactate consumed by apoptotic^{R} cell lines likely feeds into the TCA cycle for cellular energetics and amino acid production. Interestingly, the ammonia profiles of the apoptotic^{R} cultures differed from that of control cultures, with a repeated transitory drop in ammonia production for both the un-fed and lactate-fed cell lines as well as an overall decrease in ammonia production. The utilization of amino acids as an energy source in the TCA cycle is likely to lead to the accumulation of ammonia. Consequently, a reduction in ammonia production suggests that the apoptotic^{R} cell lines may be obtaining a lower relative fraction of their TCA energy requirements from amino acids compared to the control cells, which is reasonable when considering that the apoptotic^{R} cells are also consuming more lactate as a carbon source. It should be pointed out however that the drop in ammonia is transitory and the apoptotic^{R} cells may still utilize amino acids as an energy source. Indeed, three amino acids, isoleucine, leucine and valine, all branched-chain amino acids were consumed more rapidly by apoptotic^{R} cell lines and this consumption was exacerbated in lactate-fed cultures (FIG. 8A-C). Since the apoptotic^{R} cell lines experienced continued growth relative to the control cell lines, these three amino acids may form building blocks for fatty acids and other proteins which are needed by the apoptotic^{R} cells and also can be catabolized as a source of energy for the TCA cycle. A somewhat similar consumption profile is also observed for methionine (FIG. 8D).

In contrast to the amino acids which are observed to decline, a few amino acids were secreted including alanine, transiently and aspartate, transiently in the control cultures. A portion of the pyruvate can be converted to alanine by alanine amino-transferase. This pathway may be more active in apoptotic^{R} cells leading to a larger rise in alanine concentration relative to the control cell lines (FIG. 8B). Aspartate, on the other hand, is the first member of the aspartate amino acid family and is formed along with alpha-ketoglurate by the conversion of TCA cycle intermediate oxaloacetate and glutamate using aspartate transminase. Its level increases only in the control cells for the first six days while being continuously depleted in the apoptotic^{R} cell lines. Since this reaction is reversible, the depletion of aspartate in the apoptotic^{R} cell lines early in the growth phase indicates a possible limitation in the reversible oxaloacetate product in the TCA cycle for apoptotic^{R} cell lines. Given that oxaloacetate also reacts with Acetyl-CoA during the initial step of the TCA cycle, an increased flux of pyruvate into Acetyl-CoA from lactate in the apoptotic^{R} cell lines may enhance the demand for oxaloacetate. In contrast, there may be less of a demand for Acetyl CoA in the control cell lines since they are not consuming lactate and thus they generate aspartate from oxaloacetate. Indeed, this lack of demand for Acetyl CoA could be driving the build-up of lactate as an alternative by-product to Acetyl CoA in the control cells over the first seven days of the cell culture. Alternatively, a lack of lactate consumption in the control cells could lead to a limitation in the supply of Acetyl-CoA and a concomitant increase in the production of aspartate.

These results are indicative of heightened TCA cycle energetics in cell lines expressing anti-apoptotic genes although an exact interpretation of the cellular reactions awaits more detailed metabolic flux analysis. Irrespective of the fate of the consumed lactate, in the present invention it has been demonstrated that apoptotic^{R} cell lines consume accumulated lactate, which in turn, aids in their increased longevity and IVCC.

If apoptotic^{R} cell lines are unique in that they can extend their longevity by consuming the accumulated lactate, then there is a possibility that these lines can be cultured in medium containing lactate or medium containing higher than standard concentrations of glucose. While growth and viability could not be supported in either type of cell line when lactate was provided as the sole carbon source, lactate consumption was observed in apoptotic^{R} cell lines when it was provided as a component, in addition to glucose (data not shown) or when lactate accumulated as a waste product. This suggests that the apoptotic^{R} cells cannot perform gluconeogenesis from pyruvate or obtain enough energy from lactate, but if glucose is provided in conjunction to lactate the cells can consume both the glucose as well as lactate accumulated or supplemented to the culture. Moreover, in medium containing high glucose (60 mM), the IVCC (and VCD and viability, FIGS. 9A-C) of apoptotic^{R} cell lines was significantly higher than that of control cell lines. This is due to the fact that, in the high glucose medium, the control cell lines amassed more than 20 mM lactate, which may have been toxic. The apoptotic^{R} cell lines on the other hand, were consuming the lactate early in the growth phase and thus able to maintain higher numbers of viable cells that translated to an increase in IVCC of 170% over control (FIG. 9C).

Since the apoptotic^{R} cell lines consumed lactate and achieved higher IVCC, production cell lines expressing proteins of interest such as therapeutic antibodies derived from apoptotic^{R} host cell lines achieved significantly higher titers compared to production cell lines derived from control cell lines. Such higher titers illustrate the potential commercial benefits of using the anti-apoptosis genes in mammalian cell lines producing therapeutic or other proteins of interest.

The present invention will now be described with reference to the following specific, non-limiting examples.

### EXAMPLES

In the following Examples, CHO cell lines over-expressing anti-apoptosis genes were analyzed in shake flask cultures for peak viable cell density, longevity, caspase-3 activation and mitochondrial membrane potential (MMP). Additionally, nutrient consumption and metabolite production in these cell lines were compared to that of control cell lines to determine if the expression of anti-apoptosis genes had any effect on the nutrient consumption and metabolite accumulation in mammalian cell culture systems.

### Materials and Methods:

### Cell Culture:

CHOK1SV cell line (Lonza Biologics, Slough, UK), designated as the Control cell line C1013A, was cultured in CD-CHO medium (Cat. No. 10743-011, Invitrogen, Carlsbad, Calif.), containing 30 mM Glucose and supplemented with 6 mM L-Glutamine (Invitrogen Cat. No. 10313-021). In some instances, another animal protein-free medium containing various concentrations including 60 mM glucose (defined as the high glucose medium) was used. Fetal Bovine serum was purchased from Hyclone Labs, Logan, Utah (Cat. No. SH30071.03). Cell cultures were monitored by a Cedex automated cell counting instrument (Innovatis, Germany). Integrated viable cell count (IVCC, cell-day/ml) was calculated using the following formula:

IVCC (d1)=[VCD (d0)+VCD(d1)]/2+VCD (d0),

where VCD=viable cell density

### Plasmid Construction:

pBUDCE4.1 vector designed to constitutively express E1B-19K (EF-1a promoter), either alone or in conjunction with Aven (CMV promoter) and has been described (Nivitchanyong et. al., 2007). The vector expressing XIAPΔ (CMV promoter) was as described in Sauerwald et al., 2002. The blank vector refers to the original pBUDCE4.1 vector.

A model antibody (Ab #1) expression vector was constructed by cloning a Heavy and a Light chain cDNA into the Glutamine Synthase (GS) expression vector (obtained from Lonza Biologics, Slough, UK, under a research license).

Generation of Apoptotic^{R} cell lines:

An exponential culture of CHOK1SV cell line was transfected with 1) pBUDCE4.1; 2) pBUDCE4.1-E1B-19K; 3) pBUDCE4.1-E1B-19K-Aven; and 4) pBUDCE4.1-E1B-19K-Aven and pCMV-XIAPΔ. Transfections were performed using Fugene (Roche, Cat. #1815075, Basel, Switzerland) according to the manufacturer's recommendation. Two days post-transfection, cells were plated in 96-well plates in growth medium containing 300μg/ml Zeocin (for transfections 1, 2 and 3 above); 300μg/ml Zeocin and 400 μg/ml Hygromycin (for transfection #4), see Table 1. About 200 antibiotic resistant clones from each transfection were expanded and analyzed for caspase3/7 activity as described below. Promising clones underwent stability testing in absence of the respective antibiotics for ten passages.

TABLE 1

| | | Genes | Clones |
|---|---|---|---|
| Cell Line | Vector(s) transfected | over-expressed | selected by |
| Control | None | None | None |
| Blank | pBUDCE4.1 | None | Zeo |
| E64 | pBUDCE4.1-E1B-19K | E1B19K | Zeo |
| EA63 | pBUDCE4.1-E1B-19K-Aven | E1B-19K, | Zeo |
| | | Aven | |
| EA112 | | | |
| EA167 | | | |
| EA190 | | | |
| EAX64 | pBUDCE4.1-E1B-19K-Aven | E1B-19K, | Zeo, Hygro |
| EAX99 | and pCMV-XIAPΔ | Aven, XIAPΔ | |
| EAX148 | | | |
| EAX197 | | | |

Initially, a control cell line was developed by transfecting the blank vector into CHOK1SV cell line and selecting zeocin-resistant colonies. These cell lines had on average about 20% lower IVCC compared to the untransfected control CHOK1SV, hence CHOK1SV was subsequently used as the control cell line in all experiments. The most promising apoptotic^{R} cell line, EAX197 as well as the control cell line were then used to develop production cell lines expressing a model antibody using the GS expression vector according to the manufacturer's recommendation. Antibody in cell culture was measured by Nephelometry (Beckman Array System).

### Shake-Flask Cultures of Apoptotic^{R} Cell Lines:

Selected apoptotic^{R} cell lines were cultured in un-fed batch mode in CD-CHO medium supplemented with 6 mM Glutamine and the requisite antibiotic selection agent. In some experiments, the batch mode cultures of apoptotic^{R} cell lines were fed with lactate to replenish the lactate they had depleted.

Additionally, select Ab-expressing cell lines were cultured in a custom formulated animal protein-free medium supplemented with 6 mM glutamine and 60 mM glucose.

### Caspase 3/7 Activity Assay:

About 3×10⁵ cells of each clone were seeded in one ml of growth medium in a 24-well plate. On day 4 (d4) post seeding, about 1×10⁵ cells were transferred in triplicate to a 96 well plate. Staurosprine (2 μM fc) was added and the cells were incubated for 16 h before assaying for caspase3/7 activity by APO-ONE kit (BD Labs). The procedure was repeated on d10, except that Staurosporine was omitted. The clones that had significantly lower caspase3/7 activity on both days were expanded into SF. The apoptotic^{R} nature of the selected clones was confirmed by flow cytometry analysis (see below) and in some instances, by measuring the mitochondrial membrane potential.

### Analysis of Apoptotic^{R} Clones by Flow Cytometry:

About 1×10⁶ cells from exponential cultures were withdrawn from each shake flask into 24 well plates, incubated with Staurosporine (2 μM fc) for 16 h, harvested and washed once in PBS. The cells were then incubated with CytoPerm (Cat. No. 2075KK, BD BioScience) to fix and permeablized them. Following a PBS wash, cells were incubated with FITC-labeled anti-caspase3 (Cat. No. 68654, BD BioScience) antibody before subjecting them to analysis by flow cytometry.

### Mitochondrial Membrane Potential (MMP) Assay:

Cells from shake flask cultures were withdrawn on d6 post-seeding and incubated with Staurosporine (5 μM fc) for 2 hours. They were subsequently washed and processed with lipophilic cationic dye, JC-1 (Cayman Labs, Ann Arbor, Mich.) as per manufacturer's recommendation. Plates were read at FL535and FL595 and the ratio was calculated.

### Western Blotting:

About 1×10⁷ cells from indicated cell lines were harvested, washed in PBS and lysed in RIPA buffer containing 1% NP-40, 120 mM Tris-Hcl, 150 mM NaCl, 0.2 mM PMSF and 1 mM EDTA. Fifty micrograms of cytosolic protein from each sample was loaded on a 4-12% NuPAGE gradient gel and separated by SDS-PAGE in a Novex system (Invitrogen). Separated protein bands were transferred onto nitrocellulose and analyzed by using: 1) an anti-mouse antibody to human E1B-19K, 1:40 dilution, (Cat No. DP-17, CalBiochem, Gibbstown, Md.); 2) an anti-Rabbit antibody to human Aven, 1:1,000 dilution, (Cat No. 612521, BD BioScience, San Jose, Calif.); and 3) an anti-mouse antibody to human XIAP (Cat No. 616713, BD BioScience). The above protocol, including the antibodies used, was optimized and standarized using known quantities (˜10 ng each) of purified E1B-19K, Aven and XIAP, purchased from commercial sources. The protein bands were visualized using ECL detection kit (GE Healthcare, Piscataway, N.J.).

### Determination of Metabolite Concentrations in Culture Medium:

Concentration of all key metabolites and waste products were determined using an YSI 2700 automated analyzer. Ammonium ion concentration was determined by flow injection analysis (Campmajo et al, 1994). Amino acids were measured by HPLC (Waters, Milford, Mass.), using a reversed-phase column (Waters).

### Example 1

### Generation and Characterization of Apoptotic-Resistant Cell Lines

A list of cell lines used in this study, the expression plasmids that were transfected in each case to generate the cell lines and the selection agent(s) used for isolating the transfectomas is shown in Table 1 above. The cell lines listed are representative of multiple clones that were generated from each transfection. The name of each cell line was derived from the anti-apoptotic genes that were transfected into the host cell line. For example, EAX197 is a cell line that was transfected with E1B-19K (E), Aven (A) and XIAPΔ (X). The notation, apoptotic^{R}, will be used to refer to a cell line that has been transfected by one or more of these anti-apoptotic genes.

All apoptotic^{R} cell lines used in this study were characterized by Western blotting, caspase 3/7 activity and mitochondrial membrane potential (data not shown). The single transfectant E64, or double transfectants EA63, EA112, EA167 and EA190, all expressed levels of E1B-91K that were above background levels observed in the control, untransfected cell line, C1013A. Of the four double transfectants, cell lines EA112 and EA167 expressed both E1B-19K (˜19 KDa) and Aven (˜55 KDa). Cell lines EA63 and EA190 expressed significant levels of E1B-19K but only very low levels of Aven. Of the four cell lines that were generated by transfecting the dual vector pBUDCE4.1-E1B-19K-Aven as well as pCMV-XIAPΔ, namely, EAX64, EAX99, EAX148 and EAX197, only EAX197 expressed detectable levels of all three proteins.

An ˜55 kDa band was observed in all lanes including in the untransfected control lane, C1013A, above the recombinant Aven band was believed to be the endogenous Aven protein as it was visualized with post-immune but not with pre-immune sera (Sauerwald et al., 2002, Chau et al., 2000). The transfected Aven gene lacks the first six amino acids, which have been shown not to impair the biological activity of the protein. Consequently, transfected Aven is slightly smaller than its endogenous counterpart. A 40 KDa band, which may represent a degradation product of Aven, was observed in all cell lines that over-expressed this protein in conjunction with E1B-19K. Degradative products of similar sizes were observed previously when Aven was over-expressed with Bcl-XL (Sauerwald et al., 2005) or Bcl-2 (Figueroa, unpublished observations). Additionally, the expression of Aven was significantly lower in triple transfectants, including EAX197 over-expressing E1B-19K, Aven and XIAPΔ.

Interestingly, the level of expression of transfected XIAPΔ was significantly lower compared to the endogenous levels of the intact XIAP protein. Note that the apparent difference in expression level between XIAP and XIAPΔ could be explained in part by the difference in antibody binding efficiency of these two forms of the anti-apoptotic protein. The apparent expression level difference between the endogenous XIAP and the transgenic XIAPΔ questions the contribution of this transfected protein in preventing apoptosis. Indeed this study suggests that there is not always a direct correlation between the expression level of a given anti-apoptotic gene and the level of apoptosis inhibition the protein can confer on a cell line.

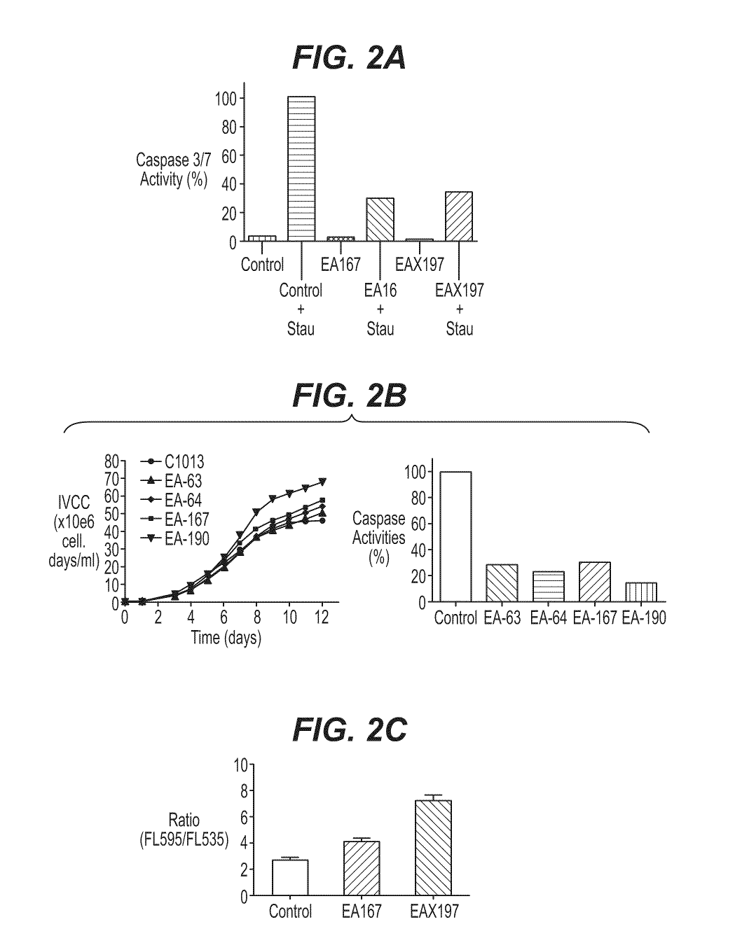

Each apoptotic^{R} cell line was examined for caspase 3/7 activity by APO-ONE as this activity provides an indication of the degree of resistance of a cell line to apoptosis. Briefly, in this assay, the profluorescent substrate Z-DEVD-R110 is cleaved by caspase 3/7 in a dose-dependent manner. Exponential cultures of EA167 and EAX197 as well as the control cell line were treated with staurosporine for 16 hours to induce apoptosis, following which caspase 3/7 was measured. The caspase 3/7 activity in staurosporine treated control cells was arbitrarily set at 100%. Under these conditions, the caspase activities of the EA167 and EAX197 cells were 30% and 35%, respectively, of the control, suggesting that these cell lines were at least partially resistant to apoptosis induction (FIG. 2A). As further evidence of the apoptotic^{R} nature of EA167 and EAX197, staurosporine-treated cultures of these two cell lines and the control cell line, were labeled with FITC-conjugated anti-caspase 3 antibody followed by analysis by flow cytometry. As can be seen from FIG. 3, all three cell lines, whether untreated or vehicle (DMSO)-treated, had 1% or less caspase 3-positive population. However, 91% of the control cell population was positive for caspase 3 following staurosporine treatment. In contrast, only 9% of the population derived from cell line EAX197 and 30% of that derived from cell line EA167 were positive for caspase 3, confirming the apoptotic^{R} phenotype of these two cell lines.

To examine if the resistance to apoptosis leads to increased longevity, multiple EA cell lines were grown in shake-flasks (batch mode) and both viable cell density (VCD) and integrated viable cell count (IVCC) were monitored. Increases in the integrated viable cell count improve the volumetric productivity of a culture, assuming that the specific productivity of the cell remains unaltered. Therefore IVCC can be used as a parameter for screening superior apoptotic^{R} cell lines that can serve as hosts for the development of production cell lines. As shown in FIG. 2B, the cell lines with lower relative caspase 3/7 activity tended to generate cell lines with higher IVCC. Although each of the EA cell lines showed reduced caspase 3/7 activity relative to the control, there was no strict correlation between IVCC and the level of caspase 3/7 that each cell line possessed. Moreover, some apoptotic^{R} cell lines were more stable than their sister clones with respect to the degree of anti-apoptotic phenotype. EA167 was chosen for further study because it possessed both potent anti-apoptotic activity and robust growth in shake flask cultures. Additionally, EA167 was the most stable cell line in this panel, expressing Aven at high levels as detected by Western blot.

Many of the signaling molecules in the apoptosis pathway interact with or reside within the mitochondria. There these proteins interact with the mitochondrial membrane and regulate apoptosis through modulation of the mitochondria permeability. Therefore, mitochondrial membrane potential (MMP) serves as another monitor of cell physiology in which to compare the characteristics of the apoptotic^{R} nature of EA167 and EAX197 versus control. As seen in the assay results of FIG. 2C, both EA167 and EAX197 had higher MMP than the control cell line.

### Example 2

### Effect of E1B-19K on Viable Cell Density of CHOK1SV

The growth profiles of double transfectants over-expressing E1B-19K in conjunction with Aven (EA167), triple transfectants over-expressing E1B-19K in conjunction with Aven and XIAPΔ (EAX197), as well as the transfectant over-expressing E1B-19K only (E64) are compared to the control (host) cell line in FIG. 4. While the peak VCD of the control cell line reached 7.7×10⁶ cells/ml and the peak VCD of E64 (expressing E1B-19K only) was 8.9×10⁶ cells/ml, that of EA167 was 1.4×10⁷ cells/ml and that of EAX197 was more than 1.5×10⁷ cells/ml, an increase of over 190% above the control cell line. The viability of EA167 and EAX197 showed a sharp decline by d8 or d9 post-seeding, presumably because of nutrient depletion, while the control and the E64 cell lines viability declined more slowly (FIG. 4B). The net effect on IVCC is shown in FIG. 4C. The IVCC of the control cell line was 4.9×10⁷ cell-day/ml, followed by that of E64 (5.2×10⁷ cell-day/ml). Clearly, the IVCC of the two apoptotic^{R} cell lines, EA167 (6.3×10⁷ cell-day/ml) and EAX197 (6.6×10⁷ cell-day/ml) were significantly higher (136% and 140% for EA167 and EAX197, respectively) than that of control cell lines.

In addition to EA167 and EAX197, several other apoptotic^{R} lines exhibited similarly low lactate production phenotype (data not shown). Additionally, wild-type cell lines with low lactate production characteristics, though rare, have been reported (Pascoe et al, 2007).

### Example 3

### Metabolic Profile of Apoptotic^{R} Cell Lines

In order to compare the physiology of the apoptotic^{R} cell lines EA167 and EAX197 which could protect against apoptosis to the control cell line in batch cultures, the levels of key nutrients and amino acids that were provided at the beginning of the batch culture, namely, glucose, glutamate and glutamine, were monitored. Additionally, the accumulation of the waste products ammonia and lactate were also monitored. Shown in FIG. 5 are the levels of these metabolites when EAX197 and EA167 as well as control cell line were cultured in batch mode in shake-flasks in commercial CD-CHO medium. As shown in FIG. 5A, glucose is rapidly depleted from the medium of all three cell lines by day-6 or day-7 post-seeding. No significant differences in glutamine depletion were discernable between apoptotic^{R} and control cell lines and glutamate levels did not vary greatly for the three cell lines across all days (data not shown). All three cell lines showed an accumulation of lactate during early cell growth phase up to day-4 (FIG. 5B). In the control cell line, lactate continued to increase up until day-6 (at which time it reached 2.8 g/L) and then a slight decrease was observed with the lactate concentration eventually reaching 1.8 g/L. This amount of lactate is expected to lower the pH of the culture and in bioreactors, where pH is controlled by the addition of bicarbonate, the osmolarity would be elevated. All these characteristics, in addition to other stress factors, could lead to a toxic environment for the animal cells. In contrast, the lactate levels in the apoptotic^{R} cell lines increased for a shorter period and then dropped rapidly from day 5 onward with the lactate being completely eliminated by day 7 (EA167) or day 8 (EAX197). Interestingly, the time point at which the lactate dropped to zero immediately preceded the time at which the VCD dropped precipitously in FIG. 5A for these two cell lines, suggesting that the loss in cell viability was due to the absence of any available glucose or lactate in the medium.

Another difference observed between apoptotic^{R} and control cell lines is the ammonia profile (FIG. 5C). The ammonia levels in EA167 and EAX197 declined slightly at day 6, at which time the decline in lactate had begun, and then ultimately increased again. However, the overall ammonia levels of the apoptotic^{R} lines were lower than that of control lines, which was surprising since the apoptotic^{R} lines reached higher IVCC and thus attained more overall cell growth compared to the control cell line (FIG. 4C).

From the above data, it is obvious that the characteristics of the apoptotic^{R} cell lines are dramatically different from that of the control cell lines. However, when multiple clones are examined under somewhat different experimental conditions, a wide spectrum of phenotypes is observed among the apoptosis^{R} transfectants. Since phenotypic variability exists in clonal isolates of the control cell line as well, it is possible to select rare apoptotic^{R} clones from control cell line by utilizing a suitable screening regimen. In this respect, Mattanovich and Borth (2006) have reviewed the application of cell sorting to isolate rare variants with desirable properties from wild-type (control) cell line.

### Example 4

### Effect of Lactate Feeding

The amount of lactate present in a cell culture at any point is dependent on the amount of lactate secreted by that cell line less the amount consumed by it. In order to determine if the apoptotic^{R} cell lines were actually consuming lactate or if the net lactate production was lower in these cell lines, cultures of all three cell lines were monitored daily and any major difference in lactate concentration between apoptotic^{R} and control cell line was eliminated by the addition of exogenous lactate. As observed previously, the lactate level in the un-fed apoptotic^{R} EA167 and EAX197 began to drop at day-5 (FIGS. 6A and 6B) with exhaustion of lactate by day-7 (EA67, un-fed) or day-8 (EAX197, un-fed). The lactate profile of the control cell line continued to increase until d6 at which time the level declined slightly for the next three days but never dropped to zero. Upon daily monitoring of the apoptosis^{R} culture, lactate was added to separate fed-batch cultures up to 2.5 g/L. at day-7 (for EA167, FIG. 6A) and 2 g/L at day-8 (for EAX197, FIG. 6B) in order for levels to be similar to that of the control cell line. Even with the addition of supplemental lactate, the lactate level dropped much more rapidly in the apoptotic^{R} cultures compared than the controls from day-6 to day-12. Indeed, the lactate level dropped from 2.5 to 0.2 g/L in the EA167 fed culture from day-6 to day-7 while the lactate decline was only from 2.7 to 2.4 g/L in the control culture over the same time period. Thus, the lactate appears to be consumed in the apoptotic^{R} cell lines at an accelerated rate compared to the control cell lines for at least 7 days following addition of supplemental lactate.

The effect of lactate supplementation on VCD and cell viability is shown in FIGS. 7A and 7B, respectively. As previously observed (FIG. 5A), the VCD of the un-fed apoptotic^{R} cell lines declined rapidly starting at day-8 (EA167) and day-9 (EAX197). This is presumably because one or more essential media components, including glucose and/or lactate, have been exhausted in these cultures. The control cell line exhibited a slow decline starting at day-7. However the lactate-supplemented cultures (EA167, lactate-fed and EAX197, lactate-fed) exhibited a much slower decline in VCD, and in one cell line (EAX197, lactate-fed), the VCD stabilized at between 8×10⁶ and 10×10⁶ cells/ml until d14 before this culture eventually succumbed. The cell viabilities shown in FIG. 7B exhibited a similar more gradual decline for the two apoptotic^{R} cultures with supplemented lactate in the media. These results suggest that the apoptotic^{R} cell lines are capable of utilizing both endogenous and supplemental lactate efficiently to retain their viable cell density and viability while the control cell line is unable to implement a similar efficient lactate consumption strategy. This difference is reflected in the IVCC of the lactate-fed apoptotic^{R} cell lines, which were nearly 180-220% higher than the un-fed apoptotic^{R} cell lines and about 235%-250% higher than the control cultures.

Most of the other nutrients and metabolites shown in FIGS. 7D-F exhibited profiles similar to that observed previously in the un-fed cultures of FIG. 5. Glucose and glutamine were rapidly consumed at early time points in the culture. The ammonia profile of the apoptotic^{R} cell lines once again exhibited a slight decline at the time at which lactate consumption began in both the unsupplemented and lactate-supplemented cultures. As in the previous experiments, the final ammonia level of the apoptotic^{R} cell lines was below control levels even though the IVCC was considerably higher in all four apoptotic^{R} cultures. The glutamate levels differed between lactate-fed and un-fed cultures with glutamate depletion being significantly increased in the fed culture as compared to the un-fed and control cell lines.

### Example 5

### Amino Acid Profile of Apoptotic^{R} and Control Cell Lines

The concentrations of each of the twenty amino acids were monitored periodically in control cell lines as well as in un-fed and lactate-fed apoptotic^{R} cell line cultures (Table II and FIG. 8). With respect to the consumption or production of amino acids, other than glutamine and glutamate, it should be first stated that none of the nine essential amino acids (phenylalanine, threonine, methionine, lysine, tryptophan, leucine, isoleucine, valine and histidine) were exhausted, as indicated in Table II. In apoptotic^{R} (un-fed) and control cell line cultures, asparagine, serine, tryptophan and cysteine (highlighted in italics) were almost completely exhausted. Interestingly, all three branched-chain amino acids, namely, isoleucine, leucine and valine (shaded light grey in Table II) were more rapidly depleted from the culture medium by apoptotic^{R} cell lines compared to the control, and this depletion is more pronounced in lactate-fed cultures (FIGS. 8A-D). In addition to glutamate, the final levels of these three amino acids were much lower from the medium in lactate-fed cultures. Other amino acids with a high percentage consumed (final concentration below 50% of their initial concentration in the medium) included tyrosine, phenylalanine, methionine (FIG. 8D) and aspartic acid (FIG. 8E).

A comparison of the amino acids that were excreted into the culture medium between the control and the apoptotic^{R} cell lines is also of interest to consider. As shown in FIG. 8E, the control cell lines accumulated aspartic acid in the culture medium over the first six days while there was a decline in the aspartic acid levels over that same time period for all four apoptotic^{R} cultures. Additionally, the apoptotic^{R} cell lines secrete significantly more alanine (FIG. 8F) over the first 6 days as compared to the control cell line, while all the cell lines consume it at later times. Also, the apoptotic^{R} cell lines produce more homocysteine over the entire cell culture in comparison to the control cell lines (FIG. 8G).

### Example 6

### Batch Culture in the Presence of High Glucose

If the apoptotic^{R} cell lines can utilize lactate, then there is a possibility that these lines may potentially thrive at glucose concentrations that could be inhibitory for control cells due to accumulation of toxic levels of lactate. Additionally, it is possible that growth of these lines could be supported by culture medium containing lactate as the sole carbon source. It was not possible to use the CD-CHO medium used in the previous experiments because such as high glucose would lead to unsustainable osmolarity and thus a new medium had to be utilized for these experiments. Therefore, we compared the growth kinetics of EAX197, which exhibited the highest sustained growth in previous experiments, to that of control cell lines in a custom-formulated animal protein-free medium containing 60 mM glucose (high glucose) or 30 mM lactate. Neither the apoptotic^{R} nor the control cell lines exhibited sustained growth in the complete absence of glucose, i.e., in the presence of lactate only (data not shown). When both cell lines were cultured in the same custom-formulated medium containing ‘high’ (60 mM) concentrations of glucose, EAX197 exhibited a higher VCD, viability and IVCC (FIGS. 9A-C) than the control cell line especially at later times in the cell culture. EAX197 cell line exhibited a smaller overall increase in IVCC (130%) over control than in the previous CD-CHO medium but the difference was still significant due to increased viability at later times. The lactate profiles of these two cell lines cultivated in ‘high’ glucose are shown in FIG. 9D. As in the case with IVCC, the lactate profiles of both lines differed somewhat from that observed in CD-CHO medium (FIG. 5D). Consistent with previous results with the CD-CHO medium, the apoptotic^{R} cell line in the custom formulation medium initiated net lactate consumption earlier (by day 2, as shown in FIG. 9D) than the control and the maximum lactate level was much lower (1.2 g/L compared to 2.4 g/L for the control cell line). The control cell exhibited a net production of lactate until day 5 at which point the control cells also began to consume the lactate in the medium. Since the CD-CHO medium is a commercial formulation with a propriety composition, it was not possible to compare exactly the medium component(s) in the two formulations in order to understand the cause of the different lactate consumption profiles in the different formulations.

### Example 7

### Productivity of an Apoptotic^{R} Cell Line in High Glucose Medium

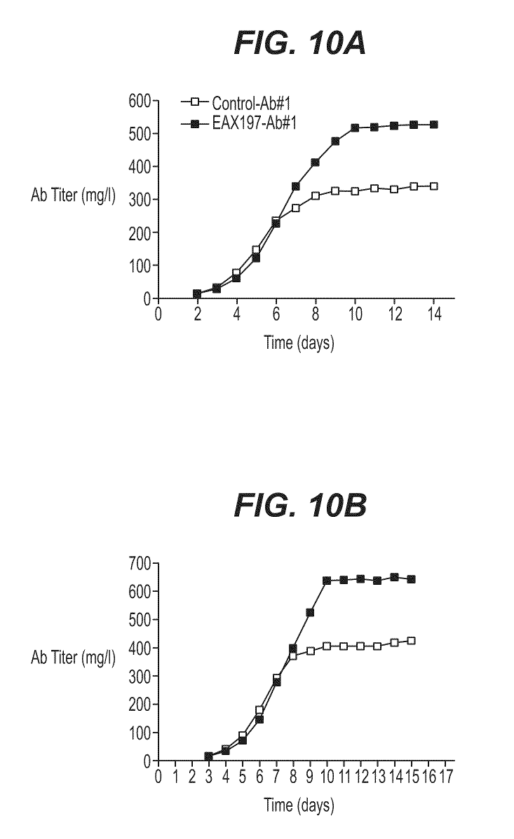

The apoptotic^{R} cell line, EAX197 along with the control cell line were subsequently used as hosts for the development of production cell lines expressing a model antibody. An equal number of clones were surveyed for each cell line and the best clone generated from each host was then compared in shake-flask-batch mode for Ab productivity. Custom formulated medium containing 60 mM (‘high’) glucose as a carbon source was used in order to compare their performance in a high glucose environment. In the standard medium (FIG. 10A), the production cell line derived from EAX197 had titers of 489 mg/L, which is 145% increase over the control cell line, which had a titer of 337 mg/L. The same cell line in high glucose medium (FIG. 10B) had titers of 687 mg/L, which is 178% increase over that of the control cell line (384 mg/L). The enhanced production was due at least in part to a 188% increase in IVCC for EAX197 versus the control cell line (data not shown).

### Example 8

### Effect of Over-Expression of E1B19K+AVEN +/−XIAPΔ in Blocking Apoptosis

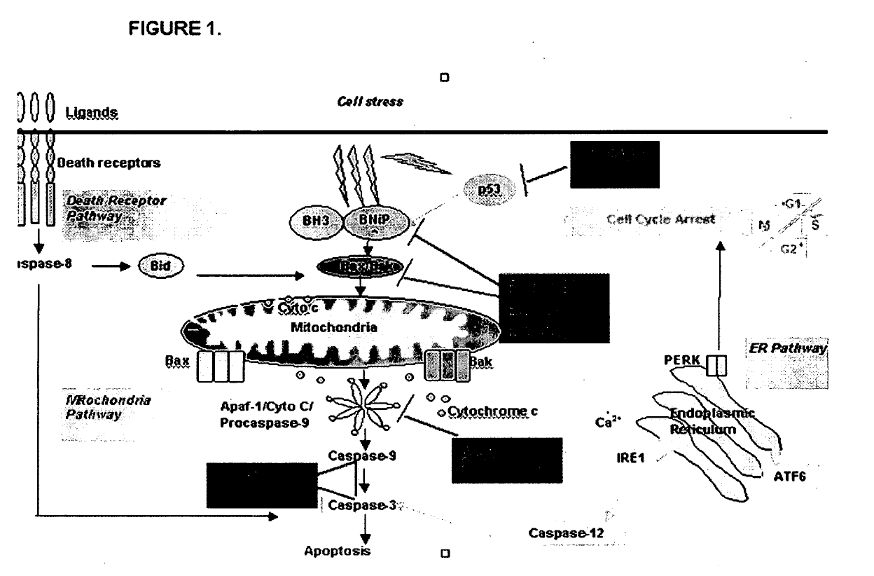

The major activators of the apoptosis cascade in mitochondria are shown in FIG. 1. In our attempt to develop an apoptotic-resistant CHO cell line, we selected three anti-apoptotic genes that act at early, mid- and late stage of apoptosis. These genes were E1B19K (E), AVEN (A) and XIAPΔ (X). CHOK1SV, the widely used transfection host cell line was transfected with E1B19K+AVEN with/without XIAPΔ. A large number of transfectomas were screened and a few clones from each transfection were selected for further study. Growth profile study of these cell lines show that they reach very high VCD and in general, their IVCC was about two-fold higher than that of control. The apoptotic^{R} cell lines lost viability by day-8 post-seeding, but regained it day-10 onwards by consuming lactate. The culture fluid of apoptotic^{R} lines expressing E1B19K+AVEN±XIAPΔ had reduced or undetectable levels of ammonia and lactate and depleted glutamate from the culture media.

### Example 9

### Use of Bcl-2d to Increase Antibody Titer

The results above demonstrate that apoptotic^{R} cell lines accumulate less lactate compared to control cells and viability of apoptotic^{R} cultures can be maintained in high glucose medium. In the following shake-flask batch experiment, an apoptotic^{R} cell line expressing Bcl-2d and antibody heavy and light chains (C2088B) was seeded at various seeding densities in standard (4.8 g/L) and high concentration of glucose (9.5 g/L). The results show that the apoptotic^{R} cell line, when seeded at 1e6cells/ml in medium containing 9.5 g/L glucose, reached peak VCD of 14e6cells/ml on day-7 post-seeding (FIG. 11A), and antibody titer of 2 g/L. In comparison, C2088B cultured under the standard conditions (0.3e5cells/ml inoculation and 4.8 g/L) had peak VCD of 7e6cells/ml and antibody titer of 1 g/L (FIG. 11B). This translates to a 200% increase in antibody titers for high glucose condition as compared to standard conditions (i.e., seeding density of 0.3e6c/ml in medium containing 4.8 g/L glucose).

The present invention now being fully described, it will be apparent to one of ordinary skill in the art that many changes and modifications can be made thereto without departing from the spirit or scope of the appended claims.

1. A method of increasing viable cell density in a fed batch eukaryotic cell culture comprising the steps of:
a) culturing a eukaryotic cell line expressing one or more heterologous apoptotic-resistant (apoptotic^{R}) genes and one or more genes of interest; and
b) maintaining a high glucose media feed during the exponential and stationary phases of the cell culture.

2. The method of claim 1 wherein the eukaryotic cell line is a Chinese Hamster Ovary (CHO) cell line.

3. The method of claim 2 wherein the CHO cell line is CHO-K1.

4. The method of claim 2 wherein the CHO cell line is CHO-K1SV.

5. The method of claim 1 wherein the eukaryotic cell line is a myeloma cell line.

6. The method of claim 5 wherein the myeloma cell line is NS0.

7. The method of claim 5 wherein the myeloma cell line is Sp2/0.

8. The method of claim 1 wherein the eukaryotic cell line is a hybridoma.

9. The method of claim 1 wherein the high glucose media feed contains about 60 mM glucose.

10. The method of claim 1 wherein the apoptotic^{R} genes comprise E1B19K and AVEN.

11. The method of claim 10 wherein the apoptotic^{R} genes further comprise XIAPΔ.

12. The method of claim 1 wherein the apoptotic^{R} gene comprises Bcl-2Δ.

13. The method of claim 1 wherein the CHO cell line consumes accumulated lactate during the cell culture exponential phase.

14. The method of claim 1 wherein the CHO cell line secretes less lactate during the cell culture exponential phase than a CHO cell line not containing one or more apoptotic^{R} genes.

15. The method of claim 1 wherein the peak viable cell density (VCD) is increased.

16. The method of claim 1 wherein the longevity of the cell culture is extended.

17. The method of claim 1 wherein the titer of the cell culture is increased.

18. The method of claim 1 wherein the integrated viable cell count (IVCC) of the cell culture is increased.

19. The method of claim 1 wherein the cellular calcium flux is reduced.

20. The method of claim 1 wherein the mitochondrial membrane potential is increased.

21. The method of claim 1 wherein the genes of interest encode an antibody heavy chain and an antibody light chain.


SEQUENCE LISTING (12 sequences)

SEQ ID NO: 1 - DNA, 660 bp, Homo sapiens
atgtcgtcccacctagtcgagccgccgccgcccctgcacaacaacaacaacaactgcgaggaaaatgagcagtctctgcccccgccggccggcctcaacagttcctgggtggagctacccatgaacagcagcaatggcaatgataatggcaatgggaaaaatggggggctggaacacgtaccatcctcatcctccatccacaatggagacatggagaagattcttttggatgcacaacatgaatcaggacagagtagttccagaggcagttctcactgtgacagcccttcgccacaagaagatgggcagatcatgtttgatgtggaaatgcacaccagcagggaccatagctctcagtcagaagaagaagttgtagaaggagagaaggaagtcgaggctttgaagaaaagtgcggactgggtatcagactggtccagtagacccgaaaacattccacccaaggagttccacttcagacaccctaaacgttctgtgtctttaagcatgaggaaaagtggagccatgaagaaagggggtattttctccgcagaatttctgaaggtgttcattccatctctcttcctttctcatgttttggctttggggctaggcatctatattggaaagcgactgagcacaccctctgccagcacctactga

SEQ ID NO: 2 - protein, 219 aa, Homo sapiens
MSSHLVEPPPPLHNNNNNCEENEQSLPPPAGLNSSWVELPMNSSNGNDNGNGKNGGLEHVPSSSSIHNGDMEKILLDAQHESGQSSSRGSSHCDSPSPQEDGQIMFDVEMHTSRDHSSQSEEEVVEGEKEVEALKKSADWVSDWSSRPENIPPKEFHFRHPKRSVSLSMRKSGAMKKGGIFSAEFLKVFIPSLFLSHVLALGLGIYIGKRLSTPSASTY

SEQ ID NO: 3 - DNA, 1089 bp, Homo sapiens
atgcaggcggagcgaggagctcggggaggccgtgggcggcggccaggccgcggccggcctggcggagatcgccacagcgagcggcccggagccgcagcggcggtagccagaggcggcggcggaggcggcggcggggacggaggcggacgccggggccgtggccgtggccggggcttccgcggcgctcgcggaggccgaggaggaggaggcgccccgcgaggcagccgccgggagccgggaggctggggcgcaggggccagcgcgccggttgaagatgacagcgatgcagagacctatggagaagagaatgatgaacagggaaattattctaaaagaaagattgtctctaactgggatcgatatcaagatattgaaaaagaggtcaataatgaaagtggagagtcacagaggggaacagatttcagtgtcctccttagctctgcaggggactcattctcacagttccggtttgctgaggagaaagaatgggatagtgaagcttcttgtccaaaacagaattcagcattttatgtggatagtgagttattggttcgagcccttcaagagctgcctctctgcctccgactcaacgttgctgccgaactggtccagggtacagttcctttagaggttcctcaggtgaaaccaaagagaactgatgatggcaagggattagggatgcagttaaaggggcccttggggcctggaggaagggggcccatctttgagctgaaatctgtggctgctggctgccctgtgttgctgggcaaagacaacccaagcccgggtccttcaagggattctcagaaacccacttccccactgcagtcagcaggagaccatttggaagaagaactagatctgttgcttaatttagatgcacctataaaagagggagataacatcttaccagatcagacgtctcaggacctgaaatccaaggaagatggggaggtggtccaagaggaagaagtttgtgcaaaaccatctgtgactgaagaaaaaaacatggaacctgagcaaccaagtacctccaaaaatgttaccgaggaagagctggaagactggttggacagcatgatttcctaa

SEQ ID NO: 4 - protein, 362 aa, Homo sapiens
MQAERGARGGRGRRPGRGRPGGDRHSERPGAAAAVARGGGGGGGGDGGGRRGRGRGRGFRGARGGRGGGGAPRGSRREPGGWGAGASAPVEDDSDAETYGEENDEQGNYSKRKIVSNWDRYQDIEKEVNNESGESQRGTDFSVLLSSAGDSFSQFRFAEEKEWDSEASCPKQNSAFYVDSELLVRALQELPLCLRLNVAAELVQGTVPLEVPQVKPKRTDDGKGLGMQLKGPLGPGGRGPIFELKSVAAGCPVLLGKDNPSPGPSRDSQKPTSPLQSAGDHLEEELDLLLNLDAPIKEGDNILPDQTSQDLKSKEDGEVVQEEEVCAKPSVTEEKNMEPEQPSTSKNVTEEELEDWLDSMIS

SEQ ID NO: 5 - DNA, 1356 bp, Homo sapiens
atgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagatagatggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgccagatgagtcaagtcagacttcattacagaaagagattagtactgaagagcagctaaggcgcctgcaagaggagaagcttatcgattga

SEQ ID NO: 6 - protein, 451 aa, Homo sapiens
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGGGLTDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMPDESSQTSLQKEISTEEQLRRLQEEKLID

SEQ ID NO: 7 - DNA, 573 bp, Homo sapiens
atggcgcacgctgggagaacagggtacgataaccgggagatagtgatgaagtacatccattataagctgtcgcagaggggctacgagtgggatgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgagccacaagtga

SEQ ID NO: 8 - protein, 190 aa, Homo sapiens
MAHAGRTGYDNREIVMKYIHYKLSQRGYEWDAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLSHK

SEQ ID NO: 9 - DNA, 1476 bp, Homo sapiens
atgtgcaataccaacatgtctgtacctactgatggtgctgtaaccacctcacagattccagcttcggaacaagagaccctggttagaccaaagccattgcttttgaagttattaaagtctgttggtgcacaaaaagacacttatactatgaaagaggttcttttttatcttggccagtatattatgactaaacgattatatgatgagaagcaacaacatattgtatattgttcaaatgatcttctaggagatttgtttggcgtgccaagcttctctgtgaaagagcacaggaaaatatataccatgatctacaggaacttggtagtagtcaatcagcaggaatcatcggactcaggtacatctgtgagtgagaacaggtgtcaccttgaaggtgggagtgatcaaaaggaccttgtacaagagcttcaggaagagaaaccttcatcttcacatttggtttctagaccatctacctcatctagaaggagagcaattagtgagacagaagaaaattcagatgaattatctggtgaacgacaaagaaaacgccacaaatctgatagtatttccctttcctttgatgaaagcctggctctgtgtgtaataagggagatatgttgtgaaagaagcagtagcagtgaatctacagggacgccatcgaatccggatcttgatgctggtgtaagtgaacattcaggtgattggttggatcaggattcagtttcagatcagtttagtgtagaatttgaagttgaatctctcgactcagaagattatagccttagtgaagaaggacaagaactctcagatgaagatgatgaggtatatcaagttactgtgtatcaggcaggggagagtgatacagattcatttgaagaagatcctgaaatttccttagctgactattggaaatgcacttcatgcaatgaaatgaatcccccccttccatcacattgcaacagatgttgggcccttcgtgagaattggcttcctgaagataaagggaaagataaaggggaaatctctgagaaagccaaactggaaaactcaacacaagctgaagagggctttgatgttcctgattgtaaaaaaactatagtgaatgattccagagagtcatgtgttgaggaaaatgatgataaaattacacaagcttcacaatcacaagaaagtgaagactattctcagccatcaacttctagtagcattatttatagcagccaagaagatgtgaaagagtttgaaagggaagaaacccaagacaaagaagagagtgtggaatctagtttgccccttaatgccattgaaccttgtgtgatttgtcaaggtcgacctaaaaatggttgcattgtccatggcaaaacaggacatcttatggcctgctttacatgtgcaaagaagctaaagaaaaggaataagccctgcccagtatgtagacaaccaattcaaatgattgtgctaacttatttcccctag

SEQ ID NO: 10 - protein, 491 aa, Mus musculus
MCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYSQPSTSSSIIYSSQEDVKEFEREETQDKEESVESSLPLNAIEPCVICQGRPKNGCIVHGKTGHLMACFTCAKKLKKRNKPCPVCRQPIQMIVLTYFP

SEQ ID NO: 11 - DNA, 513 bp, Homo sapiens
atgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggatacttttgtggaactctatgggaacaatgcagcagccgagagccgaaagggccaggaacgcttcaaccgctggttcctgacgggcatgactgtggccggcgtggttctgctgggctcactcttcagtcggaaatga

SEQ ID NO: 12 - protein, 233 aa, Homo sapiens
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK